(19) United States
(12) Patent Application Publication
(10) Pub. No.: US 2003/0020877 A1
(43) Pub. Date: Jan. 30, 2003

(54) Correcting large visual axis offset errors

(76) Inventor: Lance R. Marrou, Orlando, FL (US)

Correspondence Address: MANELLI DENISON & SELTER PLLC, 7th Floor, 2000 M Street, N.W., Washington, DC, 20036-3307, US

(21) Appl. No.: 10/207,119

(22) Filed: Jul. 30, 2002

Related U.S. Application Data
(60) Provisional application No. 60/308,129, filed on Jul. 30, 2001.

(51) Int. Cl. A61B 3/00

(52) U.S. Cl. 351/246

(57) ABSTRACT

A method is provided to correct visual axis offset errors. Such errors in the visual axis offset are large if they significantly affect the patient's vision. The visual axis offset is the difference between the visual axis and the reference axis, commonly the pupillary axis. Although correcting the visual axis offset error may be most often performed by refractive surgery, the visual axis offset error may also be corrected in spectacles or contacts lenses. A benefit of correcting large visual axis offset errors secondary to patient vision is aesthetic appeal. Significant visual axis correction may require patient training. Although this may not compensate entirely for amblyopic vision, it may alleviate part of the dysfunction. Another benefit of correcting larger visual axis errors is providing the patient with a larger area of better vision within the aperture.

This application is based on and claims priority from U.S. Provisional Application No. 60/308,129 filed on Jul. 30, 2001, the entirety of which is expressly incorporated herein by reference.

### BACKGROUND OF THE INVENTION

1. Field of the Invention

The invention primarily relates to corneal refractive surgery and, more particularly, to a method of correcting large visual axis offset errors. The present invention also relates to lenses such as spectacles or contacts that correct large visual axis offset errors. This invention may be useful in improving visual acuity, contrast sensitivity, glare/halo reduction, and amount of good vision available through the aperture.

2. Background

The human visual system is a complex optical system. The aperture through which the patient sees is not usually aligned with the visual center. This means that the eye does not maximize the window around the patient's best vision. However, an in-depth review of the optical system reveals that such a misalignment of the pupillary axis (the center of the aperture) with the visual axis (the visual or gaze center) provides the patient with an overall improvement. This is due to the physiological location of the rods and cones on the retina.

The shape of the eyeball itself provides another axis: the optical axis. In the rare case that the eyeball is actually spherical, the location of the optical axis is difficult to discern. However, in the usual case of an elliptical eye, the optical axis can be determined from the knowledge of the shape. The optical axis fits along the major axis of the ellipsoid that would coincide with the cornea. Obviously, the cornea and the globe have different shapes, but only the corneal shape applies to the patient's visual system.

For the practical application of the present invention or similar invention in refractive surgery and lens design, the optical axis or the pupillary axis can be called the reference axis. FIG. 1 shows an exemplary cross-section of a cornea illustrating the pupillary, visual, and optical axes. The visual axis offset is the difference between the visual axis and the reference axis. This difference is commonly measured as an angular difference, or as a difference in the projected radial position. See FIG. 2 for an exemplary plan view of a cornea illustrating the pupillary, visual, and optical axes in a Cartesian coordinate system of projected radial distances.

If a change in the location of the visual axis could improve the patient's vision or could effect an aesthetic improvement, then there exists a visual axis offset error. Improving the patient's vision generally refers to the visual acuity, but may also improve the contrast sensitivity, reduce glare and halos, and increase the amount of aperture through which the patient has good vision. An aesthetic improvement may let the patient gaze in a more pleasing direction, one closer in line with the pupillary center, such that it does not look so much like the patient is looking away from the target.

### SUMMARY OF THE INVENTION

An object of the invention is to correct large visual axis offset errors using corneal refractive surgery. The visual axis offset is the change from the pupil center to the visual axis. A visual axis offset error is an error where the location of the visual axis cannot be determined or where a change in the location of the visual axis might improve the quality of vision for the patient. The error of the visual axis offset may be relative to the pupil center, optical axis, or some other axis. Any visual axis offset error that contributes to a deficiency in the patient's vision may be considered large, or significant. This object is shown in FIG. 3 in 45.

Another object of the invention is to correct large visual axis offset errors using spectacles or contact lenses. This correction, however, will likely occur on a macro level rather than the micro level capability of refractive surgery. The lens method of correction of the visual axis tends to be a patient training issue, although it might not require training. In this respect, training the patient refers to assisting and supporting the patient in adjusting to the new visual axis. This object is shown in FIG. 3 in 50.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, in which:

FIG. 1 shows an exemplary cross-section of a cornea illustrating the pupillary, visual, and optical axes.

FIG. 2 shows an exemplary plan view of a cornea illustrating the pupillary, visual, and optical axes in a Cartesian coordinate system of projected radial distances.

FIG. 3 shows correction of large visual axis offset errors, in accordance with the present invention. Two exemplary paths are shown; one for correcting the visual axis offset error by using refractive surgery, and one for correcting the visual axis offset error by using lenses.

FIG. 4 shows correction of large visual axis offset errors, repeated small adjustments until the final visual axis offset is achieved, in accordance with the present invention. Two exemplary paths are shown; one for correcting the visual axis offset error by using refractive surgery, and one for correcting the visual axis offset error by using lenses.

### DETAILED DESCRIPTION OF ILLUSTRATIVE EMBODIMENTS

Large visual axis offset errors from a reference axis can impair visual acuity, increase the likelihood of glare and halos, reduce contrast sensitivity, reduce the amount of good vision available within the aperture of the human eye, and decrease the aesthetic appeal of a person's visage. Thus, correcting large visual axis offset errors is important to a patient suffering from any or all of the above symptoms.

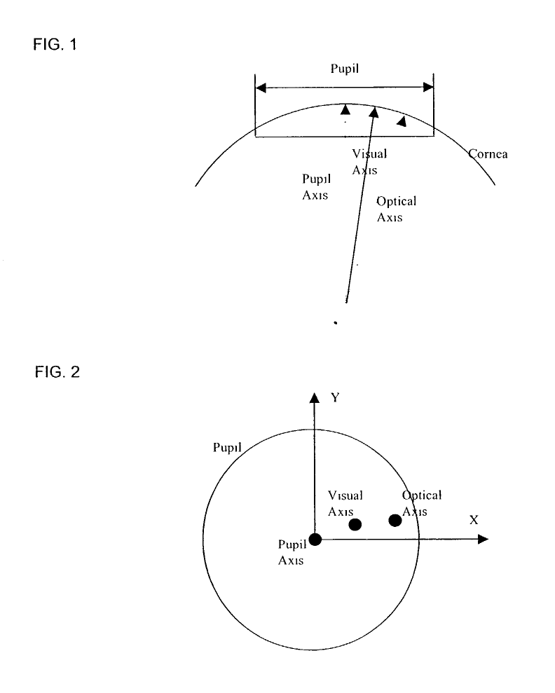

A visual axis offset is measured from the visual axis to a reference axis. The reference axis is usually the pupillary axis because that is the easiest to determine and the measure. Another choice for the reference axis is the optical axis, or geometric axis of the globe of the eye. The visual axis usually defines the gaze center, where the patient is most likely to find his or her best vision and thus look. FIG. 1 shows an exemplary cross-section of a cornea illustrating the pupillary, visual, and optical axes, when they are not coincident. It is possible that the axes (one or all) are coincident with each other. Even in that event, however, there still might be a visual axis offset error. Although the offset is, in effect, zero, a better offset for that patient might be nonzero.

FIG. 2 shows an exemplary plan view of a cornea illustrating the pupillary, visual, and optical axes in a Cartesian coordinate system of projected radial distances. This figure shows the axes from the top, typically the way in which videokeratometers, wavefront analyzers, and refractive surgery laser systems view the eye. In FIG. 2, the distance between the pupillary axis (as the reference axis) and the visual axis is the visual axis offset. In this example, the offset is a projected radial distance in the X-Y plane. From FIG. 1, the visual axis offset between the same axes could be measured as an angle, or even arc length.

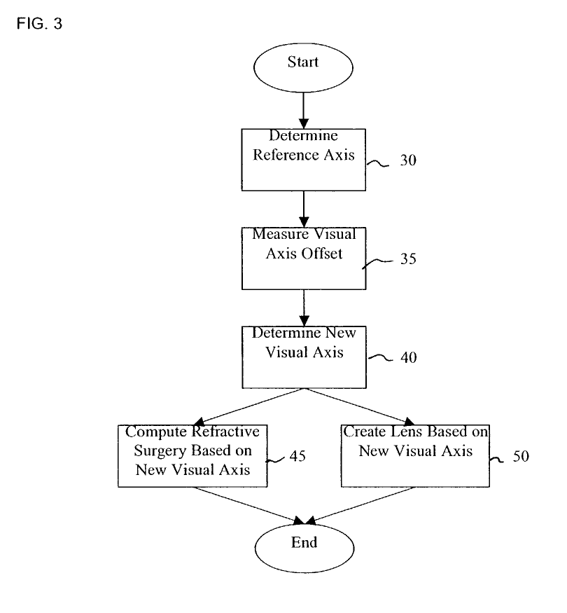

FIG. 3 provides an outline of the method for correcting large visual axis offset errors. This method has two paths, which differ only in one step, where the visual axis offset is applied to the patient. The first step 30 is to determine the reference axis. Typically, the reference axis is the pupillary axis, but it may also be the optical axis.

The next step 35 is to measure the visual axis offset. At this point, the method of measuring the visual axis offset is also determined. Example methods are projected radial distance, angle, and arc length; although others may easily be recognized that do not fundamentally differ from the present invention.

Step 40 is to determine the new visual axis. The new visual axis is the newly targeted visual axis that is expected to provide the patient some benefit. As mentioned previously, this could improve the patient's visual acuity, improve contrast sensitivity, reduce glare and halos, increase the amount of aperture through which the patient has good vision, and increase the aesthetic appeal of the patient.

In the case where the visual axis offset is in error due to an irregular corneal surface, asymmetric astigmatism, small or irregular optical zone, or similar ailment that is correctable in refractive surgery such as in step 45, it is expected that the treatment will provide improved visual acuity (likely both uncorrected and best corrected postoperatively), improved contrast sensitivity, and a reduction in glare and halos, although the benefits are not limited to this. The new visual axis may not be readily apparent postoperatively, and some patient training may be required to fully realize the benefits.

In the case where the visual axis offset is in error due to a physical or neurological defect, it is expected that the treatment will provide a reduction in glare and halos, increase the aperture through which the patient has good vision, and increase the aesthetic appeal of the patient, although the benefits are not limited to this. The new visual axis may not be readily apparent postoperatively, and some patient training may be required to fully realize the benefits. More so in this case than in the previous case, the treatment may be a newly prescribed lens such as in step 50.

A physical defect (such as naturally occurring visual axis offset errors) may be more easily and readily corrected in refractive surgery, although the lens method may also be applicable. A patient receiving such treatment may likely feel discomfort until they have adjusted to the new visual axis.

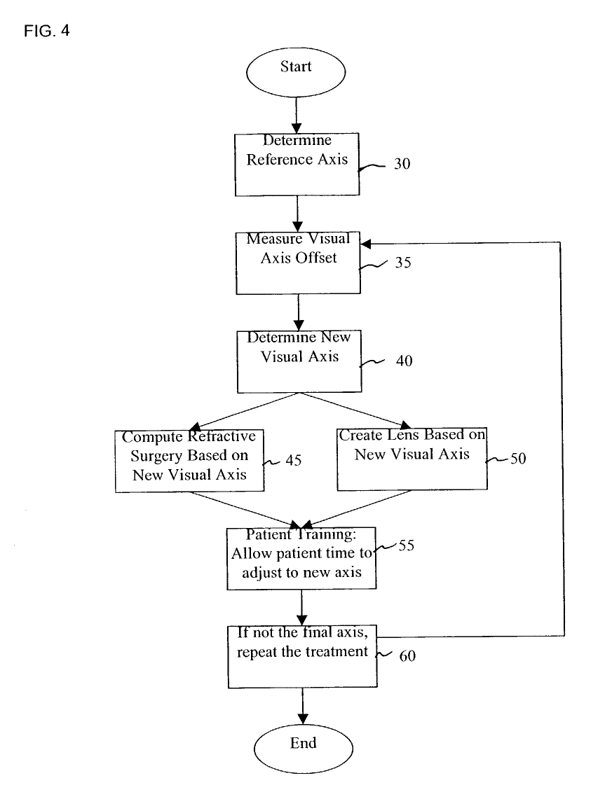

The present invention may be used in small steps, correcting the patient a little at a time until the desired visual axis offset is reached. This approach is further illustrated in FIG. 4. In this approach, step 55 allows the patient time to adjust to the new visual axis. Step 60 prescribes and applies a new treatment and the steps are repeated until the visual axis is achieved or until the treatment can no longer continue (the patient or clinician may be satisfied prior to achieving the final visual axis offset).

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A method of correcting visual axis offset errors, said method comprising:
determining a reference axis;
measuring an actual visual axis;
determining a targeted visual axis; and
applying said visual axis offset correction corresponding to a difference between said actual visual axis and said targeted visual axis, to the patient.

2. The method of correcting visual axis offset errors according to claim 1, wherein:
said method is performed during refractive surgery.

3. The method of correcting visual axis offset errors according to claim 1, wherein:
said method is performed on spectacles for the patient.

4. The method of correcting visual axis offset errors according to claim 1, wherein:
said method is performed on contact lenses for the patient.

5. The method of correcting visual axis offset errors according to claim 1, wherein said reference axis comprises:
a pupillary axis.

6. The method of correcting visual axis offset errors according to claim 1, wherein said reference axis comprises:
an optical axis.

7. The method of correcting visual axis offset errors according to claim 1, wherein said visual axis offset comprises:
a projected radial distance.

8. The method of correcting visual axis offset errors according to claim 1, wherein said visual axis offset comprises:
an angular difference.

9. The method of correcting visual axis offset errors according to claim 1, wherein said visual axis offset comprises:
an arc length.

10. The method of correcting large visual axis offset errors according to claim 1, wherein:
said targeted visual axis is divided into smaller adjustments; and
a plurality of corrections are made to the patient, each correction achieving a visual axis offset closer to said desired targeted visual axis.

11. Apparatus for correcting visual axis offset errors, comprising:
means for determining a reference axis;
means for measuring an actual visual axis;
means for determining a targeted visual axis; and
means for applying said visual axis offset correction corresponding to a difference between said actual visual axis and said targeted visual axis, to the patient.

12. The apparatus for correcting visual axis offset errors according to claim 11, wherein:
said apparatus is adapted for use in refractive surgery.

13. The apparatus for correcting visual axis offset errors according to claim 11, wherein:
said apparatus is adapted to shape spectacles for the patient.

14. The apparatus for correcting visual axis offset errors according to claim 11, wherein:
said apparatus is adapted to shape contact lenses for the patient.

15. The apparatus for correcting visual axis offset errors according to claim 11, wherein said reference axis comprises:
a pupillary axis.

16. The apparatus for correcting visual axis offset errors according to claim 11, wherein said reference axis comprises:
an optical axis.

17. The apparatus for correcting visual axis offset errors according to claim 11, wherein said visual axis offset comprises:
a projected radial distance.

18. The apparatus for correcting visual axis offset errors according to claim 11, wherein said visual axis offset comprises:
an angular difference.

19. The apparatus for correcting visual axis offset errors according to claim 11, wherein said visual axis offset comprises:
an arc length.

20. The apparatus for correcting large visual axis offset errors according to claim 11, wherein:
said means for determining a targeted visual axis divides the distance to the targeted visual axis into a plurality of smaller adjustments; and
a plurality of corrections are made to the patient, each correction achieving a visual axis offset closer to said desired targeted visual axis.
